(19) United States
(12) Patent Application Publication
(10) Pub. No.: US 2011/0313047 A1
(43) Pub. Date: Dec. 22, 2011

(54) DELIVERY SYSTEM FOR HIGH CONCENTRATION OF CERAMIDE

(75) Inventor: Marc CORNELL, Jackson, NJ (US)

(73) Assignee: L'OREAL S.A., Paris (FR)

(21) Appl. No.: 13/112,021

(22) Filed: May 20, 2011

Related U.S. Application Data
(60) Provisional application No. 61/356,366, filed on Jun. 18, 2010.

(51) Int. Cl. A61K 8/92; A61Q 19/08

(52) U.S. Cl. 514/627

(57) ABSTRACT

The present invention is directed to a delivery system capable of solubilizing high concentrations of ceramide, the system containing: (a) at least one C1-C4 monoalcohol; (b) at least one C6-C22 fatty acid and/or alcohol; and (c) from about 5 to about 14% by weight, based on the weight of the composition, of at least one ceramide.

### CROSS-REFERENCE TO RELATED APPLICATION

This application claims the benefit of the filing date of U.S. Provisional Application No. 61/356,366, filed Jun. 18, 2010, the contents of which are incorporated by reference.

### BACKGROUND OF THE INVENTION

The present invention relates to a novel delivery system for carrying high levels of ceramides. The delivery system is based on a combination of at least one C1-C4 monoalcohol and at least one C6-C22 fatty acid and/or alcohol, wherein the non-aqueous delivery system allows high levels of ceramides to be solubilized therein.

Ceramides are lipids which are naturally found in human skin. Their function within the skin is to reduce water loss by forming a protective barrier thereon. By strengthening the skin's capacity to retain water, ceramides serve to support and sustain skin's youthful appearance. Ceramides are known, however, to be difficult to solubilize at high concentrations.

Thus, it is an object of the present invention to provide a delivery system capable of solubilizing high concentrations of ceramides.

### BRIEF SUMMARY OF THE INVENTION

The present invention is directed to a delivery system capable of solubilizing high concentrations of ceramide, the system containing:

(a) at least one C1-C4 monoalcohol;
(b) at least one C6-C22 fatty acid and/or alcohol; and
(c) from about 5 to about 14% by weight, based on the weight of the composition, of at least one ceramide.

The present invention is also directed to a method of making a delivery composition capable of solubilizing high concentrations of ceramide involving the steps of:

(a) providing at least one C1-C4 monoalcohol;
(b) providing at least one C6-C22 fatty acid and/or alcohol;
(c) providing from about 5 to about 14% by weight, based on the weight of the composition, of at least one ceramide; and
(d) mixing (a)-(c) to form the composition.

### DETAILED DESCRIPTION OF THE INVENTION

Other than in the operating examples, or where otherwise indicated, all numbers expressing quantities of ingredients and/or reaction conditions, are to be understood as being modified in all instances by the term “about”.

It has been surprisingly discovered by the inventors that a combination of a C1-C4 monoalcohol with a C6-C22 fatty acid and/or alcohol yields a synergistic effect in terms of ceramide solubility.

Alcohols

The monoalcohol present in the delivery systems according to the invention contains 2 to 4 carbon atoms in a straight or branched chain, and may thus be chosen from ethanol, propanol, isopropanol, butanol, and tert-butanol. Ethanol will preferably be chosen.

The monoalcohol is advantageously present in an amount of from about 10 to about 60% by weight, based on the total weight of the system.

Fatty Acids and/or Alcohols

Suitable fatty acids for use in the present invention include those corresponding to formula (I)

R1CO—OH (I)

in which R 1CO is an aliphatic, linear or branched acyl group containing 6 to 22 carbon atoms and 0 and/or 1, 2 or 3 double bonds. Typical examples are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and the technical mixtures thereof obtained, for example, in the pressure hydrolysis of natural fats and oils, in the reduction of aldehydes from Roelen's oxosynthesis or in the dimerization of unsaturated fatty acids. A particularly preferred fatty acid for use in the present invention is isostearic acid.

Suitable fatty alcohols for use in the present invention include those corresponding to formula (II)

R1-OH (II)

in which R1 is an aliphatic, linear or branched group containing 6 to 22 carbon atoms and 0 and/or 1, 2 or 3 double bonds. Typical examples are caproyl alcohol, caprylyl alcohol, 2-ethylhexanoyl alcohol, caprilyl alcohol, lauryl alcohol isotridecanoyl alcohol, myristyl alcohol, palmityl alcohol, palmitoleyl alcohol, stearyl alcohol, isostearyl alcohol, oleyl alcohol, elaidyl alcohol, petroselyl alcohol, linoleyl alcohol, linolenyl alcohol, elaeostearyl alcohol, arachyl alcohol, gadoleyl alcohol, behenyl alcohol and erucyl alcohol and the technical mixtures thereof obtained, for example, in the pressure hydrolysis of natural fats and oils, in the reduction of aldehydes from Roelen's oxosynthesis or in the dimerization of unsaturated fatty acids. A particularly preferred fatty alcohol for use in the present invention is isostearyl alcohol.

The fatty acid and/or alcohol is advantageously present in the delivery system of the present invention in an amount of from about 35 to about 90% by weight, based on the weight of the system.

The ratio by weight of C6-C22 fatty acid and/or alcohol to C1-C4 monoalcohol is typically from 10:1 to 1:2, and all ratios by weight in between.

Ceramide

The ceramides used in particular in the compositions of the present invention can be of natural origin or synthetic, and may be of type II (for example N-oleoyldihydrosphingosine), of type III (for example N-stearoylphytosphingosine), of type IV (for example N-(alpha-hydroxybehenoyl)dihydrosphingosine) or of type V (for example N-(alpha-hydroxypalmitoyl)dihydrosphingosine).

From these simple ceramides, it is possible, in addition, to use complex ceramides which can have properties similar to those of the simple ceramides. In particular, sphingolipids such as oligoglycoceramides (gangliosides), monoglycoceramides (cerebrosides), acylmonoglycoceramides, and hydroxyacylmonoglycoceramides may be used. Sphingophospholipids such as sphingomyeline may also be used.

These simple or complex ceramides can be of vegetable origin, such as, for example, the wheat glycoceramides sold by the company ARD or a mixture of glycolipids (containing glycoceramides, phospholipids and triglycerides) sold under the trade name CERAMIDE VEGETAL by the company INOCOSM.

With respect to ceramides of natural origin, they typically are employed as mixtures of various types of ingredients with a more or less substantial ceramide content and whose composition is difficult to control. Furthermore, these mixtures are subject to bacterial contamination. Their preservation is very difficult to manage. When they are of animal orign, there is in additional risk of contamination by the agent responsible for ESE (bovine spongi-form encephalopathy).

Therefore, a particularly preferred ceramide for use in the present is one of synthetic origin corresponding to formula (II):

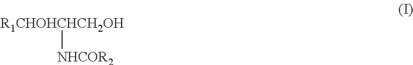

in which: R₁ denotes a C11 to C21 alkyl or alkenyl radical; R₂ denotes a C1-C19 hydrocarbon radical, which is linear and carries one or more ethylenic unsaturations, and in particular one or two, or a mixture of C11-C19 hydrocarbon radicals, which are linear, saturated or carry one or more ethylenic unsaturations, and in particular one or two, in which the proportion of saturated radicals cannot exceed 35%, these compounds being in the form of a racemic mixture of the erythro and threo diastereoisomers in the erythro:threo proportions of 85:15 to 60:40.

The ceramide will typically be present in the composition of the present invention in an amount of from about 5 to about 14% by weight, based on the total weight of the delivery system.

The present invention will be better understood from the examples which follow, all of which are intended for illustrative purposes only and are not meant to unduly limit the scope of the invention in any way.

### EXAMPLES

The following examples were prepared by combining the cited ingredients in a beaker, with mixing, for approximately 2 hours.

| | Ex. 1 | Ex. 2 | Ex. 3 |
|---|---|---|---|
| Isostearic | 95.5% by weight | 0% by weight | 44% by weight |
| acid | | | |
| Denatured | 0% by weight | 93% by weight | 44% by weight |
| alcohol | | | |
| Ceramide (*) | 4.5% by weight | 7% by weight | 12% by weight |
| Total | 100% by weight | 100% by weight | 100% by weight |
| appearance | Cloudy/hazy | Cloudy/hazy | Clear/transparent |

(*) 2-oleamido-1,3-octadecanediol commercially available under the tradename MEXANYL GX from CHIMEX.

As can be seen from the above data, the combination of isostearic acid plus monoalcohol yields a synergistic increase in the amount of ceramide which can be solubilized.

What is claimed is:

1. A delivery system capable of solubilizing high concentrations of ceramide comprising:
(a) at least one C1-C4 monoalcohol;
(b) at least one C6-C22 fatty acid and/or alcohol; and
(c) from about 5 to about 14% by weight, based on the weight of the composition, of at least one ceramide.

2. The system of claim 1 wherein (a) is denatured alcohol.

3. The system of claim 1 wherein (b) is isostearic acid.

4. The system of claim 1 wherein (c) is 2-oleamido-1,3-octadecanediol.

5. The system of claim 1 wherein (b) and (a) are employed in the system in a ratio by weight of from about 10:1 to about 1:2, respectively.

6. A method of making a delivery composition capable of solubilizing high concentrations of ceramide comprising:
(a) providing at least one C1-C4 monoalcohol;
(b) providing at least one C6-C22 fatty acid and/or alcohol;
(c) providing from about 5 to about 14% by weight, based on the weight of the composition, of at least one ceramide; and
(d) mixing (a)-(c) to form the composition.

7. The method of claim 6 wherein (a) is denatured alcohol.

8. The method of claim 6 wherein (b) is isostearic acid.

9. The method of claim 6 wherein (c) is 2-oleamido-1,3-octadecanediol.

10. The method of claim 6 wherein (b) and (a) are employed in the system in a ratio by weight of from about 10:1 to about 1:2, respectively.
